(19) United States
(12) Patent Application Publication
(10) Pub. No.: US 2016/0045193 A1
(43) Pub. Date: Feb. 18, 2016

(54) WOUND RETRACTOR

(71) Applicant: Applied Medical Resources Corporation, Rancho Santa Margarita, CA (US)

(72) Inventors: John Coleman Thress, Rancho Santa Margarita, CA (US); Thierry Kodsi, Rancho Santa Margarita, CA (US)

(21) Appl. No.: 14/825,639

(22) Filed: Aug. 13, 2015

Related U.S. Application Data
(60) Provisional application No. 62/037,501, filed on Aug. 14, 2014.

(51) Int. Cl. A61B 17/02; B29C 47/00

(52) U.S. Cl. CPC A61B 17/0293; A61B 17/0281; B29C 47/0028; B29C 47/003; B29C 47/0064; B29C 47/0066; A61B 2017/0225; A61B 2017/00955

(57) ABSTRACT

A wound retractor comprising an outer ring, an inner ring, and a flexible sheath extending therebetween is described, wherein the inner ring has the strength, rigidity and bounce back characteristics of a solid injection-molded ring yet is manufactured by extrusion techniques described herein with a 20-30% reduction in materials, resulting in reduced manufacturing costs.

### CROSS REFERENCE TO RELATED APPLICATIONS

This application claims the benefit of U.S. Application No. 62/037,501, filed Aug. 14, 2014, the entire disclosure of which is incorporated by reference.

### BACKGROUND

1. Field

This invention generally relates to medical devices and, more specifically, to a wound retractor having an improved inner ring.

2. Discussion of the Related Art

Wound retractors/protectors have been described in U.S. Pat. Nos. 7,650,887; 7,727,146; 7,883,461; 7,913,697; 8,235,054, and 8,267,858, the disclosures of which are hereby incorporated by reference in their entireties. The basic components of such wound retractors include an outer ring or rings, which may be flexible or rigid, an inner ring, and flexible sleeve attached at either end to the outer and inner ring.

In use, the inner ring of the wound retractor is compressed for insertion through an incision. Once placed, the inner ring requires a particular “bounce back” force to ensure that while the device is retracted and in use during surgery, the inner ring does not collapse, which would result in loss of retraction.

Because of the particular requirements for proper function of the inner ring, the inner ring is typically manufactured by injection molding and is fully dense. Such rings have the requisite stiffness and bounce back force to function properly in a wound retractor. To save manufacturing time and costs, it is desirable to manufacture the inner ring by extruding the ring material into tubes and then welding the tube to form a ring. This manufacturing process allows for material reduction, by introducing lumen(s) for example, thereby saving costs, but may produce unsatisfactory results when used in a wound retractor. Hollow tubing or tubing with one or more lumens are not generally as stiff as a fully dense tube of the same size, nor do they have the requisite bounce back to function in a wound retractor.

What is needed, therefore, is an inner ring that can be manufactured using extrusion techniques with reduced material costs yet still maintains the rigidity and bounce back of a fully dense injection-molded ring, so that the final product costs less but maintains retraction when the wound retractor is deployed.

### SUMMARY OF THE INVENTION

A wound retractor incorporating an inner ring manufactured using extrusion techniques with reduced material costs yet still maintaining the rigidity and bounce back of a fully dense injection-molded ring is described. Although a ring with void spaces or lumens generally has less strength and bounce-back force than a fully dense molded ring, it was surprisingly discovered that a ring could be fashioned by more cost effective extrusion techniques, providing a substantial reduction in material costs, while maintaining the superior features of the fully dense ring, provided a stable internal wall structure is maintained within the ring cross-section. Moreover, it was determined that such rings provide the same performance as fully dense rings when incorporated into wound retractors.

The inventive, more cost-effective wound retractor comprises a longitudinal axis defining an instrument access channel extending from a proximal end to a distal end, an outer ring, an inner ring, wherein the inner ring has a teardrop-shaped cross section, a first lumen, a second lumen, and a stable internal wall structure separating the first lumen from the second lumen, and a flexible, tubular sheath extending between the outer ring and the inner ring. In some embodiments, the outer ring comprises an annular axis around which the outer ring is rotatable. Optionally, the outer ring includes surfaces that are easy to grip and turn, allowing the user to roll the flexible ring over itself to provide the sheath with a radial retraction force sufficient to stretch an incision to a desired diameter. In some embodiments, the outer ring may be rolled over itself to provide the sheath with a radial retraction force sufficient to seal the sheath against the edges of a wound incision.

Also described is a method for manufacturing an inner ring for use in a wound retractor, comprising the steps of providing a die having teardrop-shaped cross-section; providing tip tooling having a first tip and a second tip, wherein the second tip has a generally circular shape having a diameter R1 with a region of concavity having a radius R2, wherein the first tip and the second tip are separated by a distance L1; extruding a thermoplastic elastomer through the die and tip tooling to thereby produce a tube having a teardrop cross-section, a first lumen, a second lumen, and a stable internal wall structure separating the first lumen from the second lumen by the distance L2; cutting the tube into a length equal to the desired circumference of the inner ring to produce a first end and a second end; and welding the first end to the second end to produce the inner ring.

In some embodiments of the method of manufacturing, the first tip has a generally triangular shape. In some embodiments, R1 is approximately 0.15 inches to approximately 0.2 inches; optionally, R1 is approximately 0.17 inches. In some embodiments, R2 is approximately 0.2 inches to approximately 0.3 inches; optionally, R2 is approximately 0.25 inches. In some embodiments, L2 is approximately 0.06 inches to approximately 0.10 inches; optionally L2 is approximately 0.06 inches.

Inner rings for wound retractors manufactured by the above methods are also described.

### BRIEF DESCRIPTION OF THE DRAWINGS

FIG. 1 illustrates an isometric view of an adjustable wound retractor.

FIG. 2 is a cross-sectional view of a wound retractor deployed in an incision.

FIG. 3 is a photograph of die and tip tooling used to produce a tear-drop shaped ring with two lumens.

FIG. 4 is a photograph of the cross-section of the ring produced using the tooling of FIG. 3.

FIG. 5 is a perspective view of a 3-D model of a new tip tooling having a concavity in the tip.

FIG. 6 is a cross-sectional view of the tip tooling of FIG. 5, shown with dimensions of one embodiment of the invention.

FIG. 7 is a cross-sectional view of the lower tip of the tooling of FIG. 5, shown with dimensions of one embodiment of the invention.

FIG. 8 is a cross-sectional view of the die and tip tooling used to produce an extruded tube having a teardrop-shaped cross section, two lumens and a stable internal wall structure, shown with dimensions of one embodiment of the invention.

FIG. 9 is a cross-sectional of the die and tip tooling used to produce an extruded tube having a teardrop-shaped cross section, two lumens and a stable internal wall structure, shown without dimensions.

FIG. 10 is a photograph of the cross-section of the ring produced using the tooling of FIG. 9.

### DETAILED DESCRIPTION

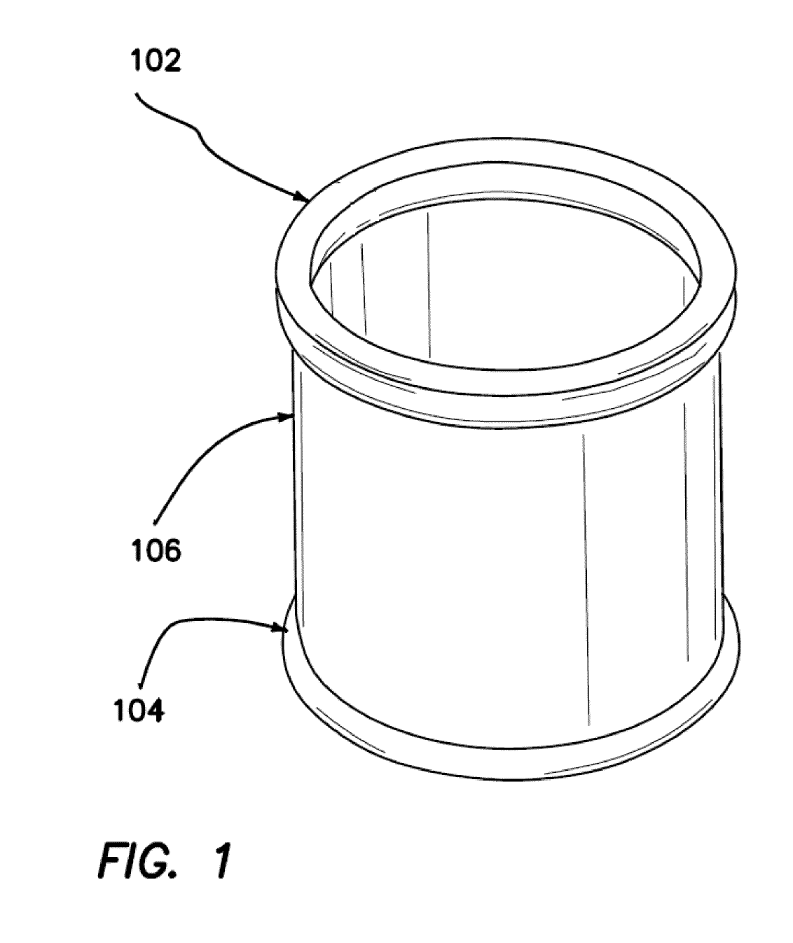

FIG. 1 illustrates an adjustable wound retractor 100 useful in a variety of surgical procedures. The wound retractor 100 includes an outer ring 102, an inner ring 104, and a distensible sleeve 106 coupling the outer ring and the inner ring. Outer ring 102 is shown as a single ring, but may also be a double ring or triple ring or multiples thereof.

The sleeve 106 may be coupled to the outer ring 102 and the inner ring 104 by heat seal, adhesive, or other means that are well known in the art. The sleeve 106 may be made of a material that is flexible and impermeable to fluids and bacteria.

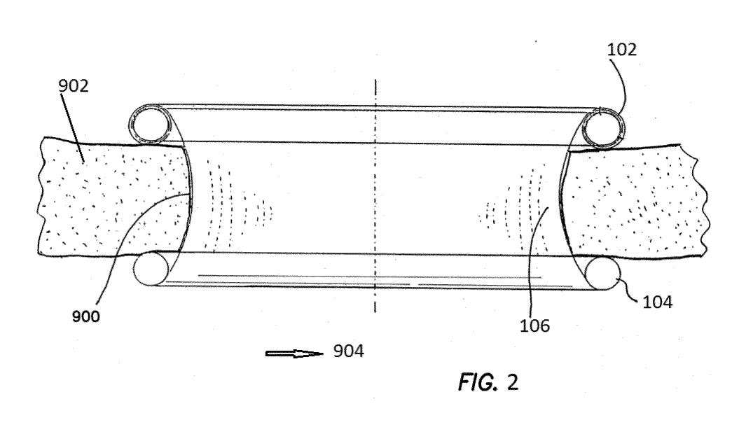

The inner ring 104 may be made of materials of sufficient hardness to retain its shape after insertion into a body cavity 904 (FIG. 2) but sufficiently flexible so as to allow the inner ring to be compressed for insertion through an incision. The materials of which the outer ring 102 is made must allow the outer ring to be turned around its annular axis as further described below. The shape of the outer ring 102 affects both its ability to grip and to provide stability during and after adjustment.

FIG. 2 illustrates the wound retractor deployed in a wound opening 900. To deploy the wound retractor, an incision in the shape of a slit is first made in the body wall 902 of a patient, such as the abdominal wall 902. The inner ring 104 is compressed and the inner ring and sleeve 106 are manually inserted into the body cavity 904 through the incision with the outer ring 102 remaining external to the body cavity. Once the inner ring 104 is within the body cavity 904, it expands around the inner surface of the incision 900 so as to be generally parallel to the outer surface of the abdominal wall 902. The sleeve 106 provides a working channel from outside the body cavity 904 to inside the body cavity.

The outer ring 102 initially rests above the abdominal wall 902 around the wound opening 900. Since the upper end of the sleeve 106 is coupled to the outer ring 102, the sleeve 106 can be drawn upwards and radially outward or inward, thereby drawing the inner ring 104 tightly against the inner surface of the abdominal wall 902. Moreover, the intermediate portion of the sleeve 106 is drawn tightly against the sides and edges of the wound opening 900, thereby retracting the adjacent tissue and producing a tightly sealed opening in the body cavity 904. The sleeve 106 contacts the entire surface of the wound 900 and protectively covers and seals it from contamination and infection. Depending on the size and depth of the incision 900, the user can roll up the sleeve 106 by gripping the outer ring 102 and rotating it until the sleeve 106 abuts the outer edge of the wound opening 900. The inner ring 104 is adapted for juxtaposition with the inner surface of the abdominal wall 902 and the outer ring 102 is adapted for juxtaposition with the outer surface of the abdominal wall. Both the inner ring 104 and the outer ring 102 are adapted for disposition relative to the incision 900 in the abdominal wall 902. The sleeve 106 is adapted to traverse the incision 900 in the abdominal wall 902.

In one embodiment of the invention, the outer ring is solid. Alternatively, the outer ring may have one or more lumens. In another embodiment of the invention, a small wire, such as a stainless steel wire, or other stiffening element is placed inside a lumen of the outer ring 102. The wire provides retraction stability to the wound retractor 100.

After surgery, the wound retractor 100 may be retrieved by grabbing the inner ring 104 and the sleeve 106 and pulling them through the wound opening 900. The use of the sleeve 106 and the ease of retracting the outer ring 102 provide higher compression between the inner and outer rings. As a result, the wound retractor 100 provides incremental adjustability to fit a wide range of incision sizes and isolates and protects the wound from bacterial infection as diseased body parts and contaminated instruments are passed through the wound.

As noted above, the inner ring of the wound retractor must be sufficiently flexible to be compressed for insertion through an incision into a body wall, but able to return to its ring shape after insertion so that it can anchor the wound retractor against the inner wall. The inner ring must also be sufficiently rigid to maintain its shape during retraction and throughout the surgical procedure. Inner rings of suitable flexibility and strength may be formed from a variety of suitable materials, including thermoplastic polyurethane elastomers, such as PELLETHANE or IROGRAN.

The inner and outer rings may be manufactured using a variety of techniques known in the art, including injection molding and extrusion. In forming a ring using the extrusion process, the plastic material is extruded as a tube, with or without lumen(s), and then welded to into a ring. Typically, the inner ring is produced as a fully dense tube, often using injection molding, so that it has the rigidity and bounce back required in a wound retractor. The present invention is directed at an inner ring that requires less material than a fully dense ring while maintaining all the functional advantages of a fully dense ring, preferably formed by extrusion rather than injection molding. Such an inner ring functions as well in a retractor as a fully dense ring, but is much less expensive to produce due to savings in materials and manufacturing.

The plastic extrusion process is known, but in brief, raw material, often called resin, is fed into the hopper of an extruder. Additives, such as colorant, may be added to the resin prior to adding it to the hopper. From the hopper, the resin is fed into the extruder barrel and propelled forward through the barrel, typically by the action of a screw mounted to a screw drive motor. As the resin moves through the barrel, it is heated until the resin reaches a molten state. The molten resin is generally filtered through a screen after exiting the barrel; from there, it is fed into the head assembly and then the tip and die tooling, which gives the tubing its final profile, including lumens, surface features, etc. Pressurized air may be used to keep void spaces, such as lumens, open until the tubing cools. The product is cooled, for example, by pulling the extruded material through a water bath.

In the present invention, a modification of the extrusion process was developed that resulted in manufacturing cost savings through a reduction in the amount of material required to produce rings while maintaining the strength and flexibility needed for the inner rings to function properly. Specifically, it was discovered that a ring having a tear-drop shaped cross-section could be produced with less material than a solid ring and still maintain the desirable attributes of a solid ring so long as the ring had a stable interior wall structure or bridge that spanned the width of the ring. Accordingly, tip and die tooling was designed in a particular geometry to introduce lumens into the inner ring while maintaining this bridge, thus maximizing material reduction while still maintaining the inner ring's required structure and force.

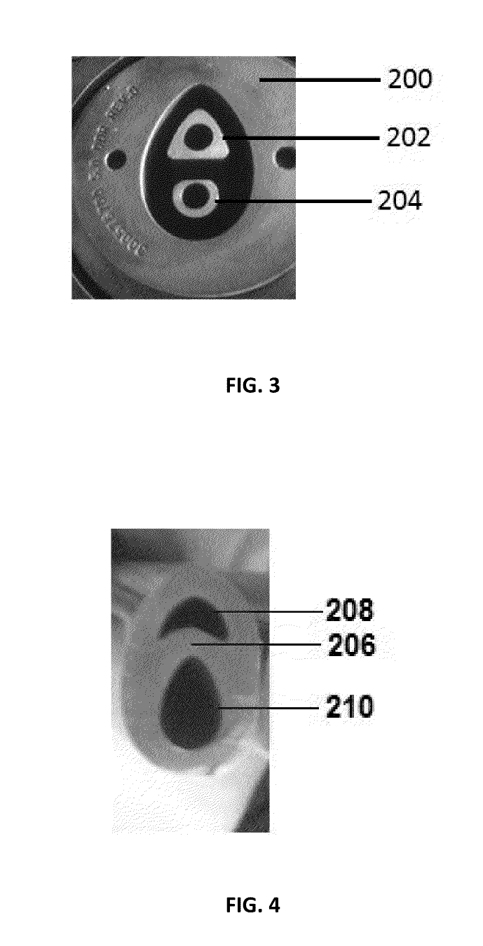

Initially, two lumens were introduced into the inner ring using a triangular tip and a circular tip. During the extrusion process, positive air pressure was introduced through tip and die tooling 200, seen in FIG. 3, having a teardrop-shaped die 218, a generally triangular upper lumen tip 202 and a generally circular lower lumen tip 204. However, when too much air pressure was applied, the lumens in the resulting ring, shown in FIG. 4, interfered with the inner ring's interior wall structure or bridge. The resulting ring had an inner wall structure 206 that was curved or “U” shaped. The upper lumen 208 was crescent-shaped, rather than the expected triangular shape, while the lower lumen 210 was distorted into a tear-drop shape rather than a circular shape. The end result was a ring that collapsed too easily, did not have the needed bounce back and was generally not suitable for functioning in a wound retractor.

To solve this problem, tip and die tooling with unique geometries were tested to identify configurations that maintain a straight inner wall structure and thus maintain the requisite rigidity of the ring. It was determined that a region of concavity introduced onto the lower lumen tip stabilized the structure of the extrudate allowing for maximimum material reduction while not affecting the inner wall structure.

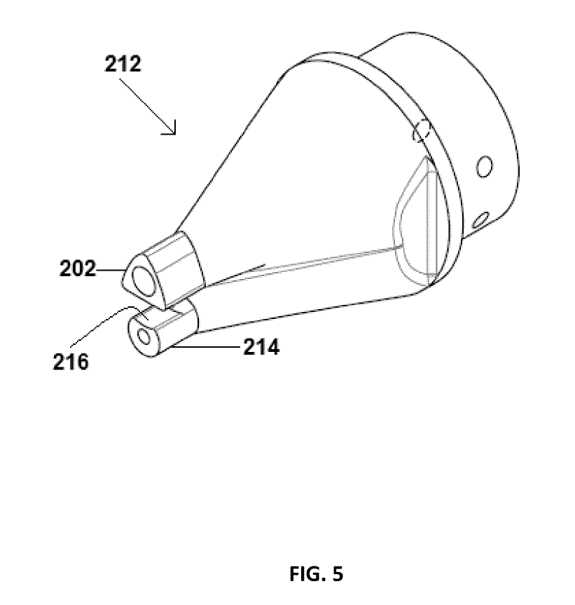

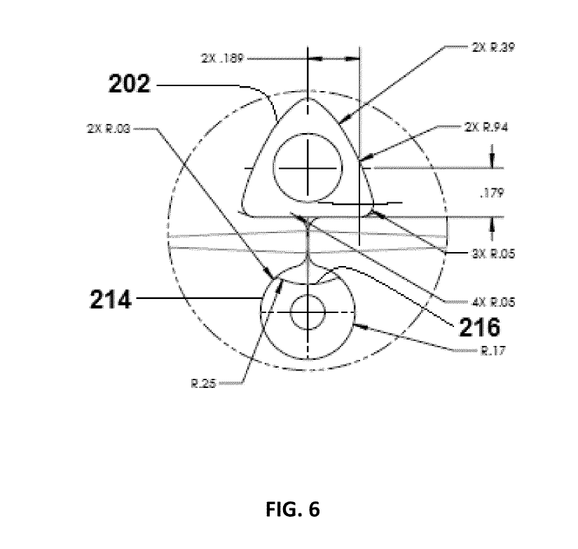

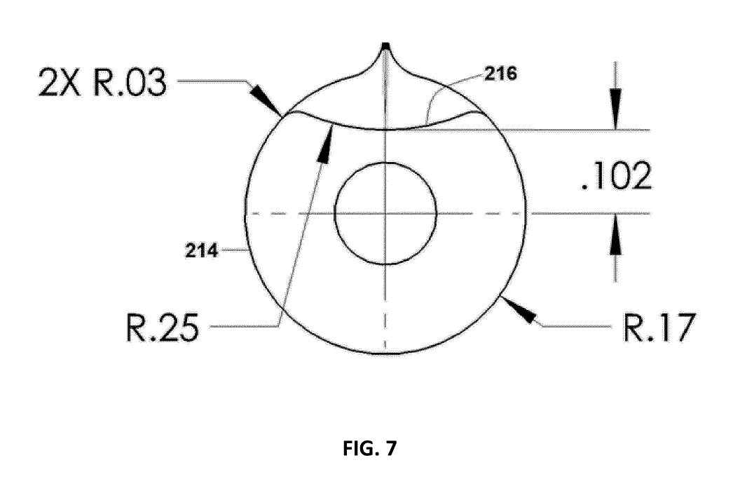

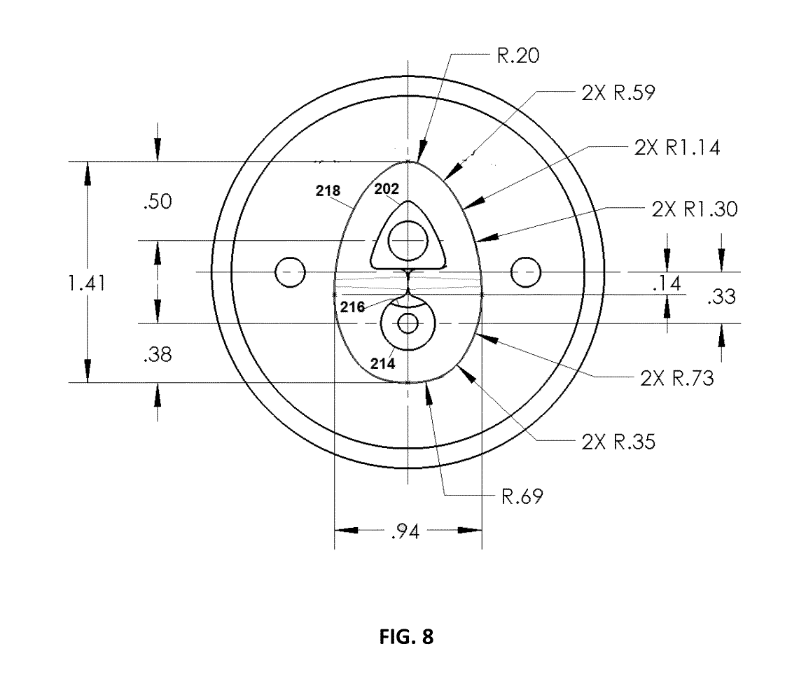

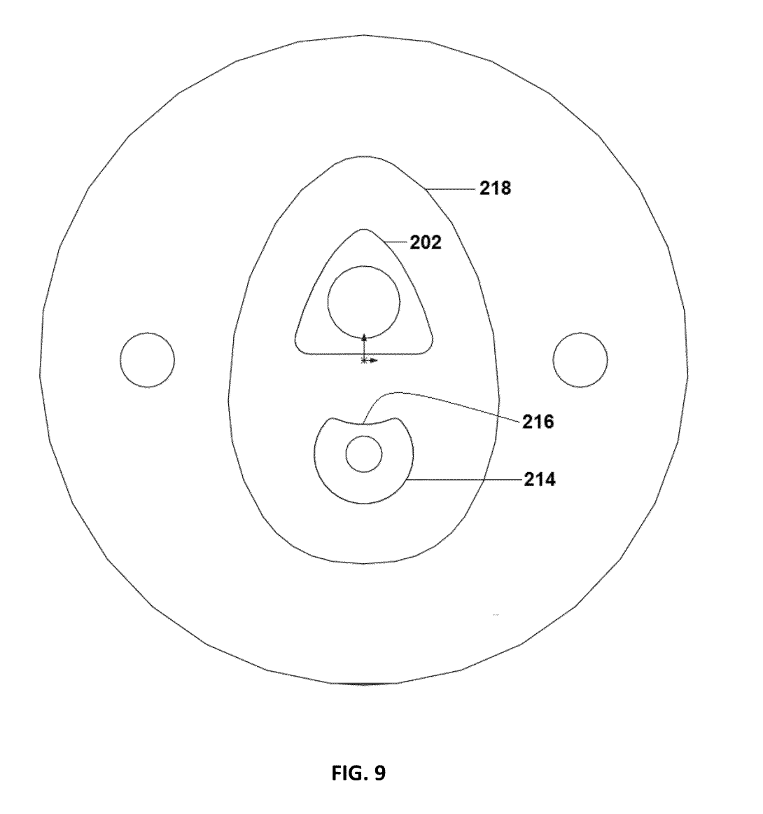

A perspective view of a 3-D model of the new tip tooling 212 is shown in FIG. 5. The new tip tooling includes an upper generally triangular tip 202 and a lower tip 214. The lower tip is generally circular with a region of concavity 216 on the edge of the lower tip closest to the upper tip. A close-up of the new tip tooling is shown with representative dimensions (in inches) in FIG. 6. A further close-up of the lower tip is shown in cross section with representative dimensions (in inches) in FIG. 7. A cross-section of the die and tip tooling is shown with dimensions (in inches) in FIG. 8.

The illustrated tip tooling will produce a tubing having a 20-25% reduction in material, although ranges from 20% to 30% may be produced by varying the process parameters of the extrusion line and the axial position of the tip relative to the die. In general, the die and tip tooling is configured to produce a stable bridge or straight inner wall extending across the cross-section of the tube, the bridge having a thickness of no less than 0.060 inches.

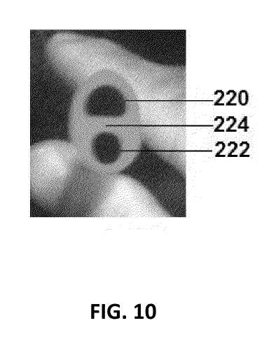

The newly designed die and tip tooling is shown in FIG. 9. This tooling was used to produce the tube shown in cross-section in FIG. 10. In this tube, the upper lumen 220 and the lower lumen 222 retain their general shape. Importantly, the interior wall structure or bridge 224 remains straight. The tubing was heat welded to form a ring and subjected to the testing described below.

In order to determine if rings having a teardrop cross-section, two lumens and a straight internal wall structure performed as well as solid injection molded rings, a number of tests were conducted. Solid rings formed using an extrusion process were used as a control. Given that both injection molded rings and rings formed by extrusion without lumen are solid, one would expect them to behave similarly in the various tests. In fact, the extruded rings with lumens and a straight inner wall structure performed better than extruded solid rings, much closer to the performance of a solid injection molded ring.

The first test was the “Inner Ring Force Test,” used to measure the bounce back force of the extruded rings (with and without lumens) compared to the injection molded solid rings. The results are shown in Table 1.

TABLE 1
Inner Ring Force Test

| Spec | Unit | Force (lbs) | Average (lbs) | Difference |
|---|---|---|---|---|
| Injection | 1 | 2.25 | 2.23 | N/A |
| Molded | 2 | 2.28 | | |
| | 3 | 2.14 | | |
| | 4 | 2.24 | | |
| | 5 | 2.25 | | |
| Extruded | 1 | 1.65 | 1.71 | 0.52 |
| without | 2 | 1.77 | | |
| lumens | 3 | 1.72 | | |
| | 4 | 1.75 | | |
| | 5 | 1.66 | | |
| Extruded | 1 | 2.06 | 2.06 | 0.18 |
| with lumens | 2 | 2.04 | | |
| | 3 | 2.08 | | |
| | 4 | 2.03 | | |
| | 5 | 2.03 | | |
| | 6 | 2.00 | | |
| | 7 | 2.05 | | |
| | 8 | 2.01 | | |
| | 9 | 1.99 | | |
| | 10 | 2.03 | | |
| | 11 | 2.10 | | |
| | 12 | 2.09 | | |
| | 13 | 2.03 | | |
| | 14 | 2.11 | | |
| | 15 | 2.10 | | |
| | 16 | 2.03 | | |
| | 17 | 2.13 | | |
| | 18 | 2.03 | | |
| | 19 | 2.05 | | |
| | 20 | 2.06 | | |
| | 21 | 2.10 | | |
| | 22 | 2.02 | | |
| | 23 | 2.04 | | |
| | 24 | 2.12 | | |
| | 25 | 2.05 | | |

Minimum Retraction Testing: This test is designed to determine if wound retractors with specific inner ring designs can sufficiently retract the minimum qualified incision compared to injection molded rings. The results are shown in Table 2.

TABLE 2
Minimum Retraction Test

| | | | Number | | | | |
|---|---|---|---|---|---|---|---|
| | | Inci- | of | | | Difference | |
| | | sion | Retrac- | Length | Width | Length | Width |
| Spec | Unit | Size | tions | (in) | (in) | (in) | (in) |
| Injection | 1 | 11 | 7 | 4.40 | 2.35 | | |
| Molded | 2 | 11 | 7 | 4.30 | 2.30 | | |
| | 3 | 11 | 7 | 4.30 | 2.35 | | |
| | | | Average | 4.33 | 2.33 | N/A | N/A |
| Extruded | 1 | 11 | 7 | 4.35 | 2.30 | | |
| without | 2 | 11 | 7 | 4.40 | 2.35 | | |
| lumens | 3 | 11 | 7 | 4.40 | 2.40 | | |
| | | | Average | 4.38 | 2.35 | 0.05 | 0.02 |
| Extruded | 1 | 11 | 7 | 4.30 | 2.30 | | |
| with | 2 | 11 | 7 | 4.40 | 2.35 | | |
| lumens | 3 | 11 | 7 | 4.35 | 2.25 | | |
| | | | Average | 4.35 | 2.30 | 0.02 | 0.03 |

Maximum Retraction Testing: The third test was used to determine if wound retractors with specific inner ring designs can sufficiently retract the maximum qualified incision compared to injection molded rings. The results are shown in Table 3.

TABLE 3
Maximum Retraction Test

| | | | Number | | | | |
|---|---|---|---|---|---|---|---|
| | | Inci- | of | | | Difference | |
| | | sion | Retrac- | Length | Width | Length | Width |
| Spec | Unit | Size | tions | (in) | (in) | (in) | (in) |
| Injection | 1 | 17 | 8 | 6.30 | 3.30 | | |
| Molded | 2 | 17 | 8 | 6.40 | 3.45 | | |
| | 3 | 17 | 8 | 6.40 | 3.60 | | |
| | | | Average | 6.37 | 3.45 | N/A | N/A |
| Extruded | 1 | 17 | 8 | 6.40 | 3.30 | | |
| without | 2 | 17 | 8 | 6.40 | 3.30 | | |
| lumens | 3 | 17 | 8 | 6.30 | 3.30 | | |
| | | | Average | 6.37 | 3.30 | 0.00 | 0.15 |
| Extruded | 1 | 17 | 8 | 6.30 | 3.30 | | |
| with | 2 | 17 | 8 | 6.40 | 3.40 | | |
| lumens | 3 | 17 | 8 | 6.40 | 3.40 | | |
| | | | Average | 6.37 | 3.37 | 0.00 | 0.08 |

Retraction Dwell Testing: Finally, the last test was used to determine if wound retractors with specific inner ring designs can sufficiently retract the nominal qualified incision for a four hour duration. The results are shown in Table 4.

TABLE 4
Dwell Test

| | | | | | | | | 4 Hour | | | |
|---|---|---|---|---|---|---|---|---|---|---|---|
| | | Incision | | Start | | 4 Hours | | Difference | | Difference | |
| | | Size | Number of | Length | Width | Length | Width | Length | Width | Length | Width |
| Spec | Unit | (cm) | Retractions | (in) | (in) | (in) | (in) | (in) | (in) | (in) | (in) |
| Injection | 1 | 11 | 8 | 6.61 | 5.73 | 6.40 | 5.56 | 0.21 | 0.17 | N/A | N/A |
| Molded | | | | | | | | | | | |
| Extruded | 1 | 11 | 8 | 6.44 | 5.66 | 6.17 | 5.24 | 0.27 | 0.42 | 0.06 | 0.25 |
| without | | | | | | | | | | | |
| lumens | | | | | | | | | | | |
| Extruded | 1 | 11 | 8 | 6.46 | 5.67 | 6.26 | 5.41 | 0.20 | 0.26 | 0.01 | 0.09 |
| with | | | | | | | | | | | |
| lumens | | | | | | | | | | | |

Results show that extruded tubing with lumens of the inventive design is more comparable to injection molded inner rings than extruded tubing without lumens. This demonstrates the inner wall running through the profile of the extruded inner ring with lumens provides more additional support than a fully dense extruded inner ring. The extruded inner ring with dual lumens of the inventive design is comparable to an injection molded inner ring and provides the requisite strength and flexibility to function in a wound retractor while providing cost savings in the manufacturing process through reduced material use.

While certain embodiments have been particularly shown and described with reference to exemplary embodiments thereof, it will be understood by those of ordinary skill in the art that various changes in form and details may be made therein without departing from the spirit and scope thereof as defined by the following claims.

What is claimed is:

1. A wound retractor comprising:
a longitudinal axis defining an instrument access channel extending from a proximal end to a distal end;
an outer ring;
an inner ring, wherein the inner ring has a teardrop-shaped cross section, a first lumen, a second lumen, and an internal wall structure separating the first lumen from the second lumen; and
a flexible, tubular sheath extending between the outer ring and the inner ring.

2. The wound retractor of claim 1, wherein the outer ring comprises an annular axis around which the outer ring is rotatable.

3. The wound retractor of claim 1, wherein the outer ring includes surfaces that are easy to grip and turn, allowing the user to roll the flexible ring over itself to provide the sheath with a radial retraction force sufficient to stretch an incision to a desired diameter.

4. The wound retractor of claim 1, wherein the outer ring may be rolled over itself to provide the sheath with a radial retraction force sufficient to seal the sheath against the edges of a wound incision.

5. A method for manufacturing an inner ring for use in a wound retractor, comprising the steps of:
providing a die having teardrop-shaped cross-section;
providing tip tooling having a first tip and a second tip, wherein the second tip has a generally circular shape having a diameter R1 with a region of concavity having a radius R2, wherein the first tip and the second tip are separated by a distance L1;
extruding a thermoplastic elastomer through the die and tip tooling to thereby produce a tube having a teardrop cross-section, a first lumen, a second lumen, and a stable internal wall structure separating the first lumen from the second lumen by the distance L2;
cutting the tube into a length equal to the desired circumference of the inner ring to produce a first end and a second end; and
welding the first end to the second end to produce the inner ring.

6. The method of claim 5, wherein the first tip has a generally triangular shape.

7. The method of claim 5, wherein R1 is approximately 0.15 inches to approximately 0.2 inches.

8. The method of claim 7, wherein R1 is approximately 0.17 inches.

9. The method of claim 5, wherein R2 is approximately 0.2 inches to approximately 0.3 inches.

10. The method of claim 9, wherein R2 is approximately 0.25 inches.

11. The method of claim 5, wherein L2 is approximately 0.06 inches to approximately 0.10 inches.

12. The method of claim 11, wherein L2 is approximately 0.06 inches.

13. An inner ring suitable for use in a wound retractor prepared by the method comprising the steps of:
providing a die having teardrop-shaped cross-section;
providing tip tooling having a first tip and a second tip, wherein the second tip has a generally circular shape having a diameter R1 with a region of concavity having a radius R2, wherein the first tip and the second tip are separated by a distance L1;
extruding a thermoplastic elastomer through the die and tip tooling to thereby produce a tube having a teardrop cross-section, a first lumen, a second lumen, and a stable internal wall structure separating the first lumen from the second lumen by the distance L2;
cutting the tube into a length equal to the desired circumference of the inner ring to produce a first end and a second end; and
welding the first end to the second end to produce the inner ring.

14. The inner ring of claim 13, wherein the first tip has a generally triangular shape.

15. The inner ring of claim 13, wherein R1 is approximately 0.15 inches to approximately 0.2 inches.

16. The inner ring of claim 15, wherein R1 is approximately 0.17 inches.

17. The inner ring of claim 13, wherein R2 is approximately 0.2 inches to approximately 0.3 inches.

18. The inner ring of claim 17, wherein R2 is approximately 0.25 inches.

19. The inner ring of claim 13, wherein L2 is approximately 0.06 inches to approximately 0.1 inches.

20. The inner ring of claim 19, wherein L2 is at least 0.6 inches.
